(19) United States
(12) Patent Application Publication
(10) Pub. No.: US 2004/0064147 A1
(43) Pub. Date: Apr. 1, 2004

(54) Introducer system having a reclosable split sheath for lead replacement

(76) Inventor: Chester L. Struble, Eijsden (NL)

Correspondence Address: MEDTRONIC, INC., 710 MEDTRONIC PARKWAY NE, MS-LC340, MINNEAPOLIS, MN, 55432-5604, US

(21) Appl. No.: 10/261,524

(22) Filed: Sep. 30, 2002

(51) Int. Cl. A61B 19/00

(52) U.S. Cl. 606/129

(57) ABSTRACT

An introducer system for performing lead replacement includes a reclosable, split sheath introducer. The sheath may preferably be a two-part sheath assembled around an implanted lead body and advanced into an existing venous entry point over an implanted lead. The sheath may optionally be provided with a distal dissecting edge to aid in advancing the introducer through fibrous tissue that may adhere or interfere with the lead. The implanted lead may be removed and a replacement lead may be introduced through the same venous entry point via the introducer. The introducer may then be withdrawn and disassembled from the replacement lead. In alternative embodiments, the introducer may preferably include a one-part sheath having curvilinear overlapping flaps that enclose the sheath lumen but may be parted to allow assembly of the introducer over an implanted lead. Another embodiment of the present invention provides a reclosable, split guide catheter.

### FIELD OF THE INVENTION

The present invention relates generally to an introducer system for the insertion of medical leads or other instruments into the venous system, and more specifically to a reclosable split sheath introducer for the removal and replacement of an implanted lead with a new lead via the same venous entry point.

### BACKGROUND OF THE INVENTION

Cardiac pacing systems commonly include an implantable pulse generator, commonly known as a pacemaker, electrically connected to the heart by at least one transvenous endocardial lead. More specifically an endocardial lead provides an electrical pathway between the pacemaker, connected to the proximal end of the lead, and endocardial tissue, in contact with the distal end of the lead. Endocardial tissue refers to a specific layer of tissue in the interior of the heart's chambers. In such a manner electrical pulses emitted by the pacemaker travel through the endocardial lead and stimulate the heart.

Endocardial leads are often placed in contact with the endocardial tissue by passage through a venous access, such as the subclavian vein, or the cephalic vein, or one of its tributaries. In such a manner, transvenous leads may advantageously be placed in contact with the heart without requiring major thoracic surgery. Rather, transvenous leads may be introduced into a vein and maneuvered therefrom into contact with the heart.

A multi-step procedure is generally required to introduce such leads within the venous system. Generally this procedure consists of inserting a hollow needle into a blood vessel, such as the subclavian vein. A wire guide is then passed through the needle into the interior portion of the vessel. The needle is then withdrawn and an introducer sheath with dilator assembly is inserted over the wire guide into the vessel. The assembly is advanced into a suitable position within the vessel, i.e. so that the distal end is well within the vessel but the proximal end is outside the patient. Next the dilator and wire guide are removed. The introducer sheath is left in position and therefore offers direct access through its hollow lumen from outside the patient to the interior of the blood vessel. A lead may be passed into the blood vessel through the introducer sheath, advanced within the cardiovascular system and ultimately positioned at a desired location for cardiac stimulation and/or sensing. For a detailed and illustrated description of these general procedures, reference is made to U.S. Pat. No. 5,713,867 issued to Morris, incorporated herein by reference in its entirety.

After the lead is satisfactorily introduced into the vein, the introducer sheath can be removed. Pacemaker leads typically have a relatively bulky connector assembly that can be 1 to 3 times wider than the lead body at the proximal end. Therefore, pacemaker leads are commonly introduced using a split or slit introducer sheath so that the sheath may be removed from around the lead by being either split or slit apart. In such a manner the introducer sheath does not have to be removed over the relatively bulky connector assembly at the proximal end of the lead. A slit introducer sheath is disclosed in the above referenced U.S. Pat. No. 5,713,867. After being split apart and removed, the introducer is typically discarded. Thus such split or slit introducers are normally single-use devices.

A situation sometimes arises that requires removal and replacement of an implanted lead. A lead may need to be acutely replaced, for example, when unacceptable stimulation thresholds are measured during an implant procedure. The implanted lead may then be removed and replaced with a different type of lead. A lead that has been implanted chronically may also need to be replaced if it has failed, or if a new type of cardiac device is being implanted which requires a different type of lead system.

A problem that arises in replacing an implanted lead is that normally the replacement lead must be implanted using the same multi-step process described above. The proximal connector assembly on a previously implanted lead prevents an introducer or guide catheter from being inserted over the implanted lead. If this were possible, the implanted lead could be removed and the new lead could be introduced through the same venous puncture site. Thus, replacement of a lead heretofore normally requires removal of the lead and repuncture of the vein at a new site for introduction of a new lead, whether the lead being replaced has been implanted acutely or chronically. Repeated venous puncture is undesirable because of the associated risks of bleeding, infection, venous wall scarring, and general venous damage.

A further problem can arise in patients who have multiple leads implanted. With the advent of multi-chamber pacemakers and implantable cardioverter defibrillators, an implantable cardiac stimulation system can include two, three or even four leads carrying electrodes to up to all four heart chambers. With continued advancement of such systems, patients may benefit from newer systems that require new or additional leads to be placed. New venous punctures or vein incisions required to replace one lead may damage other functional leads still residing within the vein.

In replacing a chronically implanted lead, scar tissue at a previous implantation site can create difficulties with lead introduction systems. Specifically the relatively tough scar tissue hinders the introduction of a dilator and introducer sheath assembly. Many times, only through use of larger incisions than are otherwise desirable is such an assembly able to be inserted.

What is needed, therefore, is an introducer system and lead replacement method that allows for a lead to be removed and replaced without requiring venous repuncture. It is also desirable that such a system overcomes the problem of scar tissue at a chronic lead implantation site.

### SUMMARY OF THE INVENTION

The present invention addresses these needs by providing an introducer system and method for use that allows a transvenous lead replacement procedure to be performed without repuncturing a vein. Aspects of the present invention allow an introducer sheath to be positioned over an existing lead such that the sheath may be advanced into an existing venous entry site. The existing lead may be removed and a replacement lead introduced through the same introducer sheath without repuncturing the vein, repositioning the introducer sheath, or requiring the use of a dilator. Thus, the risks associated with venous repuncture and the potential for damaging other leads that may already be present in a vein during a lead replacement procedure are avoided. Furthermore, the lead replacement may be performed in fewer steps, which translates into a reduction of the operation duration.

The present invention is realized by providing an introducer system featuring a sheath that opens and closes longitudinally so that it may be positioned over an implanted lead. The introducer includes a handle at a proximal end and a generally tubular, thin-walled sheath extending between a proximal and distal end having an inner lumen. In one embodiment, the sheath is formed from two elongated parts. Each part has a greater than semi-circular cross-section, i.e. a cross section that is generally in the shape of a major arc, such that when the two parts are assembled, they overlap to form a central lumen.

When used for lead replacement, one part may be positioned lengthwise along an exposed portion of an implanted lead with the second part positioned overlaying the first part along its entire length. The second part is rotated relative to the first part such that the two parts enclose the lead body circumferentially. The two parts overlap forming a fluid-resistant seal. The inner diameter of the overlapping parts is large enough to enclose the lead body but not tightly conform to the lead body. The lead body may easily slide through the introducer sheath so that the lead may be advanced or withdrawn without encountering undue friction.

In an alternative embodiment, the sheath includes one part that is generally circular in cross-section but may be opened along a longitudinal slit by parting two flexible, curved, overlapping flaps. The flaps may be parted wide enough to allow the sheath to be slipped over a lead body. When released, the flaps will reform to a generally circular cross-section with overlapping edges such that the sheath encircles the lead body and the overlapping edges form a fluid-resistant seal.

In one embodiment, the sheath is provided with a dissecting edge at its distal end. The dissecting edge may be beveled, eccentrically beveled, or scalloped and may have a tapered diameter. The dissecting edge may be further enhanced by altered material properties that increase the strength or rigidity of the distal end of the sheath for effective force transfer to the dissecting edge. For example, material processing may be performed to create a stronger, stiffer distal end or the use of braiding, coils, or other reinforcing structures formed from metal or high durometer plastics may be incorporated to reinforce and stiffen the distal end. The dissecting edge aids in the insertion of the introducer sheath over an implanted lead body and into the venous entry site by cutting through scar tissue that may be adhering to the chronically implanted lead.

A method for using the reclosable introducer sheath provided by the present invention involves exposing an implanted lead at its venous entry site and assembling the introducer sheath around the exposed lead body. The introducer is then advanced over the lead body into the vein such that the distal end of the introducer extends a sufficient distance into the vein, while the proximal open end of the sheath, through which the lead body now extends, remains outside of the vein.

The implanted lead may then be withdrawn from the vein by retracting the lead through the introducer. With the introducer still positioned in the vein, a replacement lead can be passed through the introducer and into the vein. Once the lead is advanced satisfactorily into the vein, the introducer sheath may be removed. Removal of the sheath is performed by first withdrawing it from the vein over the lead body, followed by opening the sheath longitudinally by separating the two pieces of a two-part sheath or parting the curving flaps of a one-part sheath, and finally removing the sheath from the lead body.

The replacement lead may then be advanced within the cardiovascular system to a final desired location. Depending on the particular type of lead and its application, a multitude of other instruments may be used in deploying the replacement lead, such as a guide wire or guide catheter. A guide wire or guide catheter, if used, may be advanced through the introducer sheath before the replacement lead to aid in guiding the lead to a final location.

In yet another embodiment, a reclosable split sheath guide catheter is provided. The reclosable guide catheter may advantageously be advanced over an implanted lead to allow repositioning of a transvenous lead.

Thus, aspects of the present invention allow a transvenous lead replacement procedure to be performed without repuncturing the vein. Furthermore, the introducer system and method for use provided by the present invention allow a lead replacement procedure to be performed in a shorter amount of time than the commonly practiced multi-step procedure for puncturing a vein and placing a guide wire, introducer sheath and dilator. By eliminating a new venous puncture during lead replacement, the formation of scar tissue that can impede the introduction of new leads into the same venous entry point can be reduced, and the risk of damaging other leads already present in the vein is minimized. Moreover, since repeated venous puncture is undesirable, the present invention reduces the associated risks of bleeding, infection, and general venous damage during lead replacement procedures.

### BRIEF DESCRIPTION OF THE DRAWINGS

FIG. 1 is a perspective view of a reclosable, split sheath introducer according to one embodiment of the present invention.

FIG. 2 is a perspective view of two sheath parts included in the introducer of FIG. 1.

FIGS. 3A through 3D illustrate the manner in which the two sheath parts shown in FIG. 2 may be assembled over an implanted lead body.

FIGS. 4A through 4C illustrate an alternative method for assembling a two-part sheath over an implanted lead body.

FIG. 5A is a proximal end view of an alternative embodiment of a two-part introducer sheath.

FIG. 5B is a proximal end view of yet another embodiment of a two-part introducer sheath.

FIG. 6A is a proximal end view of an introducer according to an alternative embodiment of the present invention.

FIGS. 6B and 6C illustrate the manner in which the introducer shown in FIG. 6A may be assembled over an implanted lead body.

FIG. 7 is a plan view of an introducer having a dissecting distal end according to one embodiment of the present invention.

FIG. 8 is a flow chart summarizing a method for using an introducer system provided by the present invention for replacing a chronically implanted lead.

FIG. 9 is a plan view of a reclosable split sheath guide catheter provided by the present invention.

FIGS. 10A and 10B illustrate a method for using the reclosable guide catheter of FIG. 9.

### DETAILED DESCRIPTION OF THE INVENTION

FIG. 1 is a perspective view of an introducer according to one embodiment of the present invention. The introducer 10 includes a handle 12 located at the proximal end 16 of a thin-walled, tubular sheath 14 that extends to a distal end 18. The introducer 10 is preferably formed from a biocompatible plastic, such as low density polyethylene. The introducer 10 may alternatively be produced from stainless steel, or other biocompatible metal, hence allowing for standard hospital sterilization processes. The sheath 14 may optionally include reinforcing fibers. For example the sheath may be provided with reinforcing fibers as generally described in the previously incorporated U.S. Pat. No. 5,713,867 issued to Morris et al.

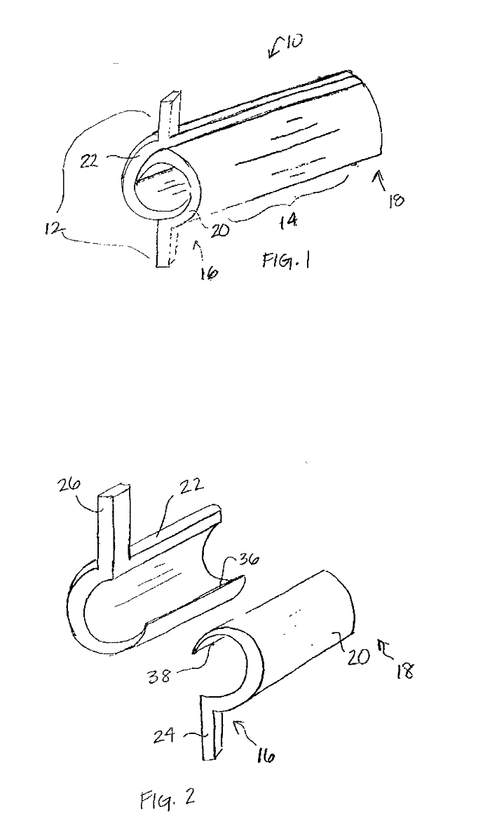

In one embodiment, the sheath 14 includes two parts 20 and 22. FIG. 2 is a perspective view of the two sheath parts 20 and 22 disassembled. Each sheath part 20 and 22 has a handle part 24 and 26, respectively, that extends radially from the proximal end 16 of the elongated sheath part 20 and 22. Each handle part 20 and 22 functions as a grip while sheath parts 20 and 22 are being positioned and rotated. Each sheath part 20 and 22 has a cross-sectional geometry that is generally in the form of a major arc such that each part partially encloses a center lumen. When assembled, as shown in FIG. 1, parts 20 and 22 will overlap, fully enclosing a center lumen.

Longitudinal edges 36 and 38 are shown in FIG. 2 to be tapered. Tapered edges 36 and 38 may ease the rotation of sheath parts 20 and 22 into an overlapping position during the assembly of introducer 10 as will be described below. Alternatively, edges 36 and 38 may be squared, beveled or otherwise.

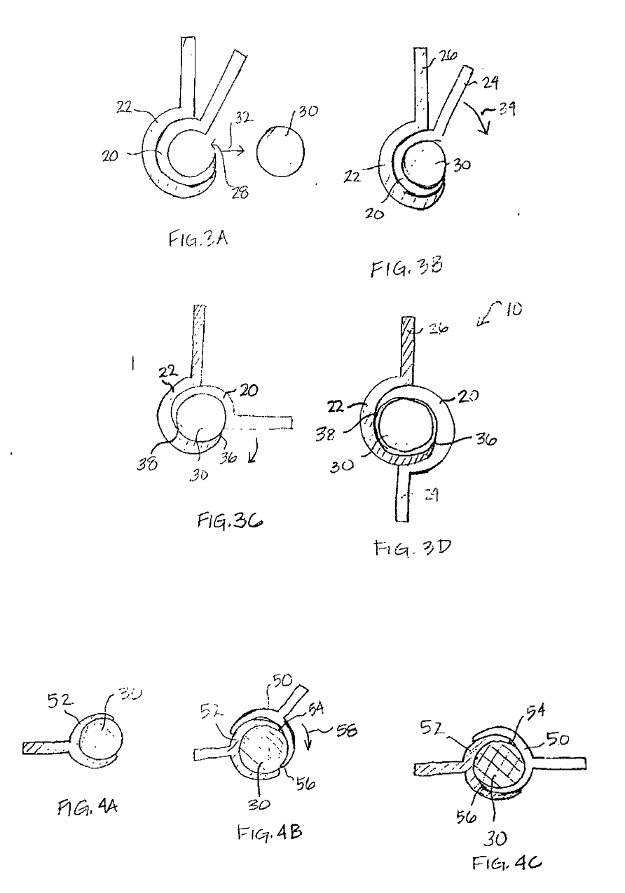

FIGS. 3A through 3D illustrate in an end view, the manner in which the two sheath parts 20 and 22 may be assembled to form introducer 10 over an implanted lead body. First, one sheath part 22 is inserted over the other sheath part 20 such that the open side of each sheath part 20 and 22 is approximately aligned. The resulting open side 28 advantageously allows the sheath parts 20 and 22 to be slipped over an implanted lead 30 as indicated by arrow 32. In order to insert one sheath part 22 over the other sheath part 20, the sheath parts 20 and 22 are flexible such that they may be opened somewhat wider than their natural radius or closed somewhat narrower than their natural radius. The sheath parts preferably possess shape memory such that they will regain their natural radius in an unstressed position.

The sheath parts 20 and 22 are shown in a partially assembled position over lead 30 in FIG. 3B. Sheath part 20 may now be rotated with respect to sheath part 22 as indicated by arrow 34. Sheath part 20 is shown partially rotated in FIG. 3C. Upon further rotation, sheath part 20 is rotated over the outside edge 36 of sheath part 22.

In FIG. 3D, sheath parts 20 and 22 can be seen in a fully assembled position. Sheath part 22 overlaps edge 38 and sheath part 20 overlaps edge 36 such that lead 30 is circumferentially enclosed within introducer 10. The inner diameter of introducer 10 is slightly larger than the outer diameter of lead 30 to allow lead 30 to easily pass through introducer 10.

Handle parts 26 and 24 are positioned opposite each other, providing a handle for aiding in advancing the introducer 10 into the venous entry site of the lead 30 and for maintaining the position of introducer 10 as the lead 30 is explanted. It is noted that handle parts 24 and 26 may be located anywhere along the outer circumference, preferably near the proximal end of the sheath parts 20 and 22. In the embodiment shown in FIG. 2, the handle parts 26 and 24 are shown located at approximate endpoints of the arc lengths that form the cross-sections of parts 20 and 22. Handle parts 24 and 26 may alternatively be located at an approximate midpoint along the arc length. Handle parts 24 and 26 are preferably positioned such that after sheath parts 20 and 22 are assembled, handle parts 24 and 26 are at 180 degrees from each other.

FIGS. 4A through 4C illustrate an alternative method for assembling a reclosable split-sheath introducer around a lead 30. In FIG. 4A, a first sheath part 52 is positioned directly over lead 30. In FIG. 4B, a second sheath part 50 is longitudinally aligned with part 52 and inserted over part 52 and lead 30 such that part 50 partially overlaps the circumference of part 52 and the lead 30. Sheath part 50 is then rotated relative to sheath part 52 as indicated by arrow 58 to circumferentially enclose lead 30. The final assembled positions of parts 50 and 52 are shown in FIG. 4C.

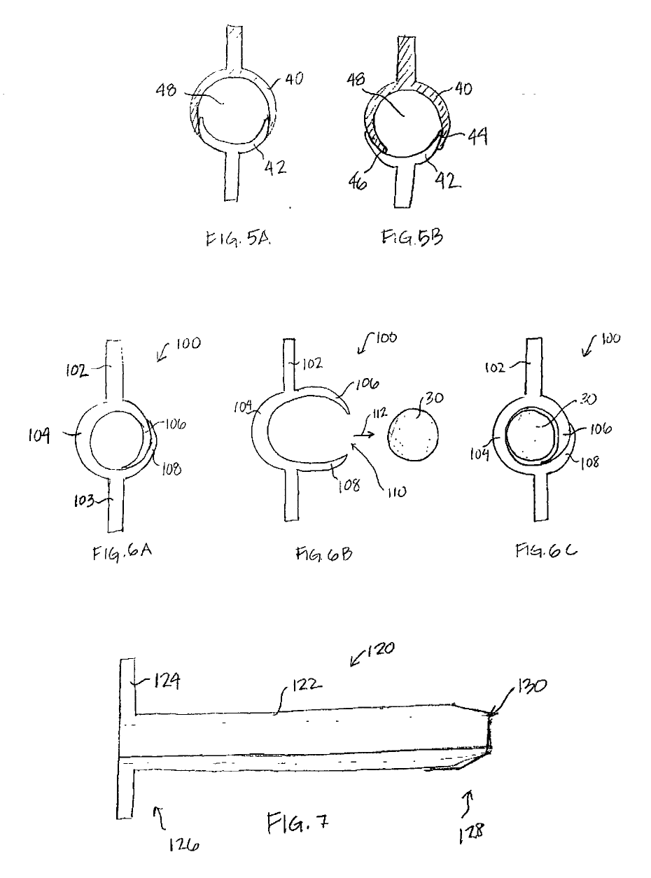

In FIG. 2, sheath parts 20 and 22 are shown having identical, or nearly identical, cross-sectional geometries that are shaped generally like a major arc. It is recognized that a two-part sheath may also be formed from non-identical parts. FIG. 5A is a proximal end view of an alternative embodiment of a two-part, reclosable split sheath. One part 40 has a cross-sectional shape generally in the form of a major arc, and a second part 42 has a cross-sectional shape generally in the form of a minor arc. The combined arc lengths of the two sheath parts 40 and 42 preferably exceed the complete circumference of the assembled sheath lumen such that the lumen 48 is fully enclosed within the assembled sheath parts 40 and 42 as shown in FIG. 5A. Preferably, the sheath parts overlap such that cohesion between the sheath parts provides a fluid-resistant seal. In FIG. 5A, sheath part 40 is shown to overlap both edges of sheath part 42. Alternatively, the proximal end view of FIG. 5B shows that sheath part 40 may overlap sheath part 42 at edge 44, and sheath part 42 may overlap sheath part 40 at edge 46.

FIG. 6A is a proximal end view of an alternative embodiment of an introducer 100 according to the present invention. In this embodiment, the introducer 100 includes a one-part, longitudinally split sheath 104. The sheath 104 is provided with curving, overlapping flexible flaps 106 and 108 extending the entire length of sheath 104. Sheath 104 is preferably formed by extruding a polymer, for example polyethylene or a fluoropolymer. Handle parts 102 and 103 extend radially outward from the proximal end of the sheath 104 and in opposite directions of each other.

FIGS. 6B and 6C illustrate the manner in which the introducer 100 may be assembled over an implanted lead 30. The flaps 106 and 108, may be opened as shown in FIG. 6B, by flexing them outward to create an opening 110 which advantageously allows introducer 100 to be slid over lead 30 as indicated by arrow 112. Once the sheath 104 is positioned over the lead 30, the flaps 106 and 108 may be released such that they reform to their original overlapping position as shown in FIG. 6C. The sheath 104 will then encircle lead 30. Cohesion of the overlapping flaps 106 and 108 will provide a fluid-resistant seal. The inner diameter of sheath 104 is sized slightly larger than the lead 30 such that the lead 30 fits within the sheath 104 and easily slides past sheath 104 when the lead 30 is retracted during explantation, and when a replacement lead is forwarded through the sheath 104 for implantation.

FIG. 7 is a side view of an introducer according to an alternative embodiment of the present invention. The introducer 120 includes a thin-walled, tubular sheath 122, which may be a one-part or two-part sheath, extending between a proximal end 126 and a distal end 128. A handle 124 is provided at the proximal end 126. A dissecting edge 130 is provided at the distal end 128. Dissecting edge 130 aids in the deployment of introducer 120 by cutting away fibrous tissue that may be encapsulating and adhering to an implanted lead. Dissecting edge 130 is preferably a relatively sharp and stiff edge that allows the introducer to cut through tissue as it is advanced forward in the venous system. Dissecting edge 130 may be formed as a beveled, eccentric beveled, or scalloped edge that may decrease in diameter such that it is tapered. Distal end 128 may be enhanced, for example, by material processing performed to create a stronger, stiffer distal end for transferring force to the dissecting edge 130. The distal end 128 may also be reinforced by braiding, coils or other reinforcing structures formed from metal or high durometer plastics in order to stiffen the distal end 128.

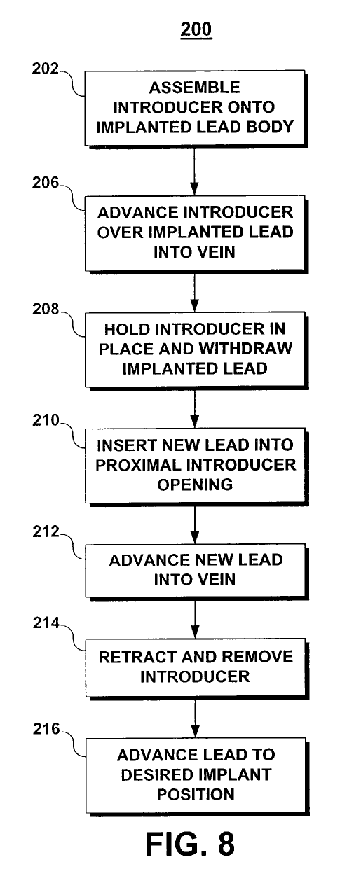

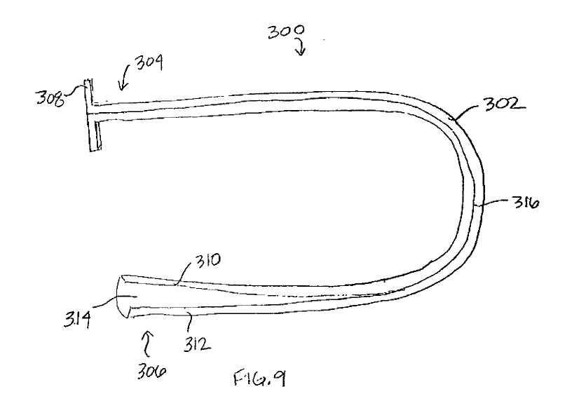

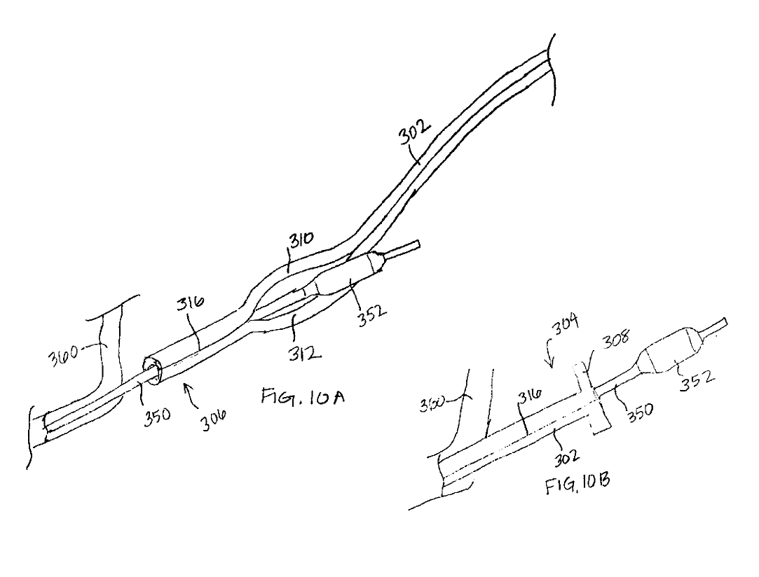

FIG. 8 is a flow chart summarizing a method 200 for using an introducer system provided by the present invention for replacing a chronically implanted lead. At step 202, the introducer is assembled onto the implanted lead body. This assembly process may generally be performed as described in conjunction with FIGS. 3A-3D, FIGS. 4A-4C, or FIGS. 6A-6C. At step 206, the introducer is advanced over the lead body into the venous entry point and into the vein. The introducer is advanced such that its distal end extends well into the vein yet its proximal end remains outside the vein.

The implanted lead may then be removed at step 208 by retracting the lead through the introducer while holding the introducer in place at the venous entry point. A new lead may be introduced at step 210 by inserting the new lead into the introducer lumen through the proximal end opening. At step 212, the new lead is advanced through the introducer and into the vein.

With the new lead successfully introduced into the vein and advanced an appropriate distance, the introducer may be removed. At step 214 the introducer is retracted from the venous entry point over the new lead body and then disassembled from the lead body. At step 216, implantation of the new lead is completed by advancing the lead to a desired implant position.

Final implantation of the lead may be aided by the use of a guide catheter, a guide wire, or a steerable stylet. If used, a guide wire or guide catheter may be passed through the introducer first after removing an implanted lead. The introducer may then be removed and the replacement lead implanted with the aid of the guide wire or guide catheter.

The reclosable, split sheath introducer provided by the present invention has the advantage of being reusable during a single implant procedure. For example, a reclosable, split introducer may be useful for repositioning leads that require the use of a guide wire or guide catheter. First, a lead may be implanted according to conventional methods with the aid of a reclosable introducer and guide wire or catheter. If the lead requires repositioning during the implant procedure, after the guide wire or catheter has been removed, the reclosable introducer may be reassembled over the lead and reinserted into the venous entry point. The lead may be removed and a corresponding guide catheter or guide wire may be reintroduced through the same venous entry point. The same lead may then be re-implanted at a new position with the aid of the guide catheter or wire without requiring a venous repuncture or the use of a new introducer.

FIG. 9 is an alternative embodiment of the present invention in which a reclosable split guide catheter is provided for use in repositioning or replacing a lead. The reclosable guide catheter 300 includes an elongated body 302 extending from a proximal end 304 to a distal end 306. A handle 308 is provided at the proximal end 304. The body 302 has a central lumen 314 that may be opened along longitudinal split 316. The body 302 is provided with curving, overlapping flaps 310 and 312, which enclose body lumen 314 in their native state. Flaps 310 and 312 may be flexed outward, as shown near distal end 306 in FIG. 9, to open the body lumen 314 to allow guide catheter 300 to be inserted over an implanted lead.

FIG. 10A is a perspective view illustrating the manner in which catheter 300 may be assembled over an implanted lead. A lead having a lead body 350 and a proximal connector assembly 352 is shown entering a vein 360. The distal end 306 is first inserted over lead body 350 by opening the distal end 306 at split 316. The catheter 300 may then be advanced onto lead body 350. As the catheter 300 is advanced, the lumen 314 is opened by parting flaps 310 and 312 at split 316 such that the catheter 300 may be inserted over lead body 350, rather than over the relatively bulky lead connector assembly 352. The guide catheter 300 is progressively opened along slit 316 as it is advanced, moving from distal end 306 to proximal end 304, and reclosed around lead body 350.

FIG. 10B illustrates the catheter 300 after it has been fully advanced into vein 360 over lead body 350. Lead body 350 and proximal connector assembly 352 now extend from proximal end 304 of catheter 300. The lead may now be repositioned with the aid of guide catheter 300. The lead may also be removed leaving the guide catheter 300 in place to allow delivery of a replacement lead.

Thus, an introducer system has been described which allows a lead replacement procedure to be performed without repuncturing a vein for placement of a new lead. Numerous variations of the described embodiments are possible for practicing the invention. Therefore, the embodiments described herein should be considered exemplary, rather than limiting, with regard to the following claims.

1. An introducer system for use with a catheter or lead comprising:
a sheath forming a lumen having a proximal end and a distal end; and
means for opening and closing the sheath to encase a catheter or lead body.

2. The introducer system of claim 1 wherein the sheath further includes a cutting means at the distal sheath end for dissecting tissue to advance the sheath over a chronically implanted lead or catheter body into a venous entry site.

3. The introducer system of claim 1 wherein the sheath comprises mating parts, each part having a proximal end and a distal end and a central lumen open on one side of each part for the full length of the sheath.

4. The introducer system of claim 1 wherein said means for closing the sheath comprises means for moving one of said mating parts relative to the other part to engageably overlap the parts forming the lumen.

5. The introducer system of claim 1 wherein said means for opening the sheath includes means for rotating one of said mating parts relative to the other part.
